(19) United States
(12) Patent Application Publication
(10) Pub. No.: US 2009/0120126 A1
(43) Pub. Date: May 14, 2009

(54) DISPOSABLE ICE BAG

(76) Inventor: Robert J. Mew, Quartz Hill, CA (US)

Correspondence Address: WEST & ASSOCIATES, A PC, 2815 MITCHELL DRIVE, SUITE 209, WALNUT CREEK, CA, 94598, US

(21) Appl. No.: 11/937,281

(22) Filed: Nov. 8, 2007

(51) Int. Cl. A61F 7/10; F25D 3/08

(52) U.S. Cl. 62/530; 607/112

(57) ABSTRACT

A disposable ice bag having at least one extension that can secure the bag to an injured area.

### BACKGROUND

1. Field of the Invention

The present disclosure relates to the field of medical equipment, particularly ice bags used in the treatment on injuries.

2. Background

Ice has long been used a treatment for musculoskeletal injuries. Applying cold to an injury reduces inflammation, slows blood flow to the area, and has an analgesic effect. Immediate application to an acute injury can even help to improve the long-term prognosis by minimizing initial inflammation. Ice is also commonly used for rehabilitating injuries and chronic pain management.

Athletes in particular make extensive use of ice in managing their injuries and conditions. Athletic trainers and coaches are often prepared at events with ice on hand in case an injury occurs. However, at athletic events, portability of gear and supplies is often necessary, and having adequate supplies that are convenient to store, use and move around can be an important consideration.

Getting the ice in contact with the injured area is critical to achieve the maximum benefit of the cold. However, conventional ice bags can be difficult to keep in place. A person with an acute injury isn't likely to be moving around very much, but someone applying ice as part of an ongoing treatment protocol may want to remain mobile without an ice bag slipping out of position.

Several products exist that can hold an ice bag securely in place. For example, the Body Glove® Deluxe Ice Wrap and other similar products use hook-and-loop closures to affix a neoprene cover over an ice bag and onto the affected body part. The “WET-ICE” Pack is another device comprising an ice bag integrated into a wrap with hook-and-loop closures to hold it in place.

Although these devices serve to hold an ice bag in place, they are not disposable. Almost any plastic bag can be used as a disposable ice bag, but they can be difficult to hold in place. Trainers often take conventional plastic bags filled with ice and use athletic tape or plastic wrap to secure the bags in place. Although the materials used in this method are disposable, the result can be cumbersome and messy. Further, if an ice bag is not sealed properly, it can leak water, which can weaken the holding strength of the wrapping material, particularly if it is adhesive or friction-based.

Other disposable ice bags exist, such as that described in U.S. Pat. No. 6,470,705 to Bride-Flynn. This patent teaches a disposable ice bag with optional ties. However, the ties alone may not effectively hold a bag in place for several reasons. It may not be possible to comfortably secure the bag in place by merely tying the straps, as taught in the Bride-Flynn patent. A knot could easily come loose, causing the bag to slip. If a user ties the straps too tight or with a tight knot, it could be difficult to readjust the straps. Further a user may want to remove the bag and refill it with ice, and having to tie and untie knots could be inconvenient.

What is needed is a disposable device that can contain ice and can be easily and securely attached to an injured body part, as well as conveniently adjusted.

### BRIEF DESCRIPTION OF THE DRAWINGS

FIG. 1 shows a perspective view of one embodiment of the present device.

FIG. 1a shows a perspective view of another embodiment of the present device having two extensions.

FIG. 1b shows a perspective view of another embodiment of the present device having three extensions.

FIG. 1e shows a perspective view of another embodiment of the present device having extensions securable by complementary protrusions and apertures.

FIG. 2 shows a perspective view of another embodiment of the present device having one extension affixable to the surface of the bag.

FIG. 3 shows a perspective view of another embodiment of the present device having a single extension that can be connected to the bag by at least one pair of protrusions and apertures.

FIG. 4 shows a perspective view of another embodiment of the present device having an elastomeric region on at least one extension.

FIG. 5 shows a perspective view of another embodiment of the present device having extensions comprising at least one pair of a strap and a loop.

### DETAILED DESCRIPTION

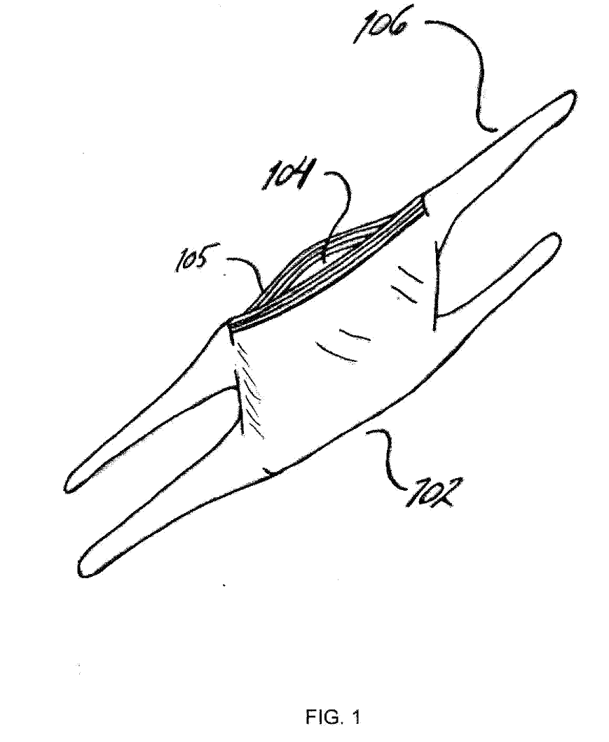

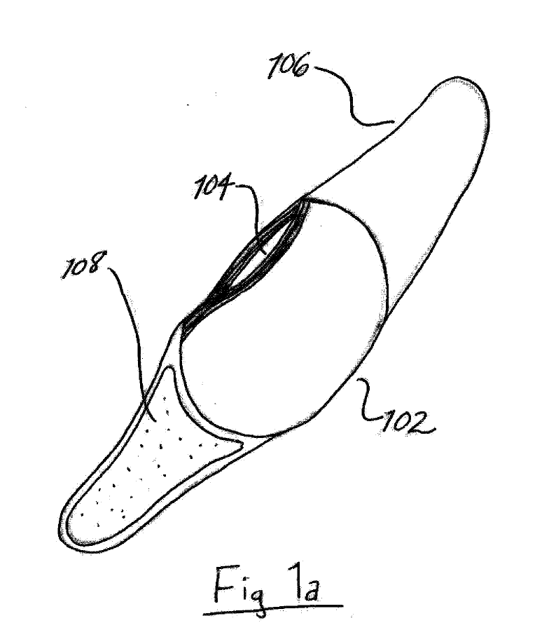

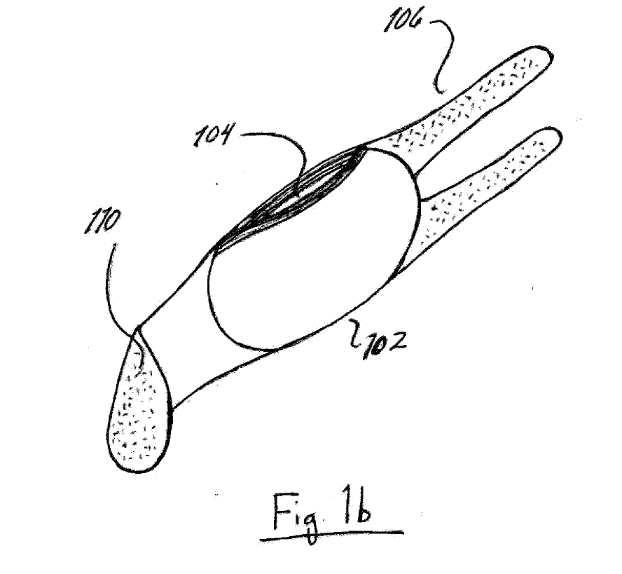

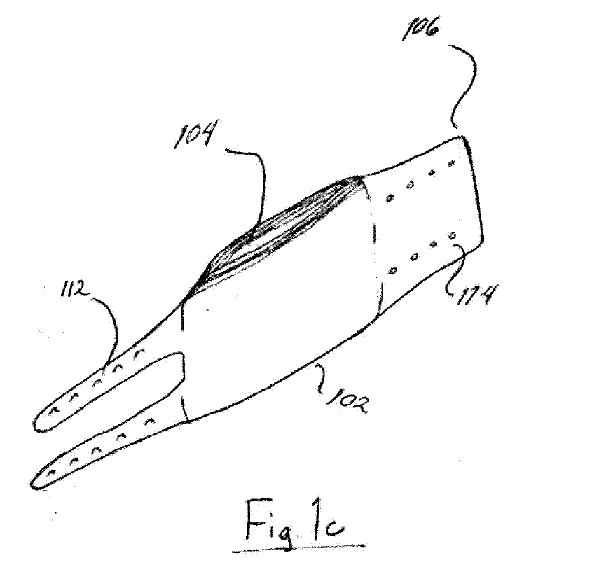

FIG. 1 shows a perspective view of one embodiment of the present device. A bag 102 can have at least one opening 104 with a closure device 105. At least one extension 106 can extend from a bag 102.

In some embodiments, a bag 102 can be made of polyurethane, polyethylene, latex-based, non-latex-based, or any other known and/or convenient disposable, waterproof material. In some embodiments, a bag 102 can be made of an air-permeable, but not water-permeable material, such as, but not limited to Tyvek®. A bag 102 can be made of a transparent, translucent, or opaque material in any known and/or convenient color and can further include any desired markings.

A bag 102 can have a square, rectangular, round, ovoid, or any other known and/or convenient planar geometry. In some embodiments, as shown in FIG. 1a, a bag 102 can have a rounded planar geometry to allow a bag 102 to more effectively contour to the surface of a body part. In other embodiments, a bag 102 can have a simpler planar geometry, such as, but not limited to a square, for ease of manufacturing or storage. A bag 102 can also be produced in a range of sizes to accommodate various body parts and/or storage considerations.

A bag 102 can be manufactured as from at least two planar surfaces of any known and/or planar geometry. In some embodiments, said planar surfaces can be joined by heat sealing, adhesive, or any other known and/or convenient method. In other embodiments, a bag 102 can be manufactured from a single planar surface that can be folded over onto itself, and the at least two of the open edges can be joined by heat sealing, adhesive, or any other known and/or convenient method.

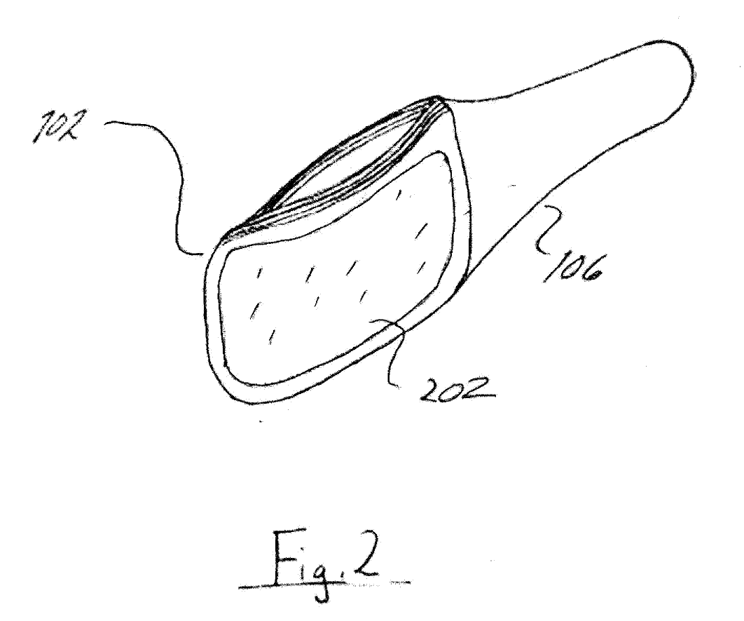

In some embodiments, as shown in FIG. 2, a bag 102 can have an adhesive, texturing, hook-and-loop material, or any other known and/or convenient material on the exterior surface of a bag 102. In such embodiments, at least one extension 106 can have additional adhesive, texturing, hook-and-loop material, or any other known and/or convenient material on at least one surface. Alternatively, at least one extension 106 cannot have any additional material on a surface, as shown in FIG. 2.

As shown in FIG. 2, an embodiment of the present device can have have only one extension 106. In some embodiments, an extension 106 can affix directly to the outer surface of a bag 102.

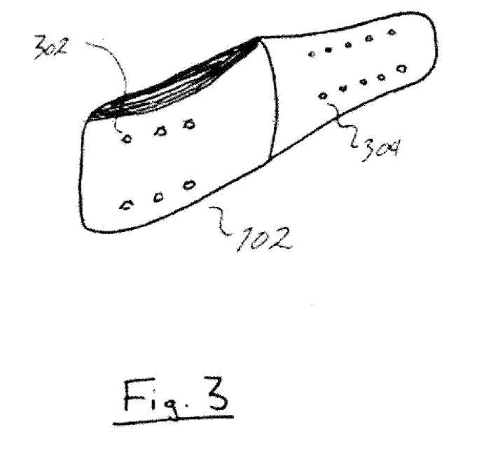

In other embodiments, as shown in FIG. 3, a bag 102 can have at least one protrusion 302 on the exterior surface of a bag 102, while at least one extension 106 can have at least one aperture 304 capable of selectively coupling with said at least one protrusion 302. In some embodiments, said at least one protrusion 302 can be integrated with a bag 102, but alternatively can be a separate protrusion attached to the exterior surface of a bag 102.

An opening 104 can be located on an edge of a bag 102, as shown in FIG. 1, or at any known and/or convenient position on a bag 102. In some embodiments, an opening 104 can be made resealable and water-tight with a closure device 105, which can be a plastic closure device, adhesive strip, or any other known and/or convenient device.

At least one extension 106 can be integrated with or attached to a bag 102. In some embodiments, as shown in FIG. 1, at least one extension 106 can be continuous with a wall of a bag 102. In alternative embodiments, as shown in FIG. 1a, at least one extension 106 can be attached to the exterior surface of a bag 102 or between sealed edges of a bag 102. In some embodiments, as shown in FIG. 1a, at least one extension 106 can comprise a pair of extensions extending outward substantially from opposite sides of a bag 102. At least one extension 106 can extend substantially perpendicularly or at any other known and/or convenient angle from an edge or surface of a bag 102.

In some embodiments, as shown in FIG. 1a, at least one extension 106 can further comprise an adhesive material 108 affixed to at least one surface of at least one extension 106. Adhesive material 108 can be capable of affixing to another adhesive material, the outer surface of a bag 102, another extension 106, or to skin, and can be hypo-allergenic and waterproof.

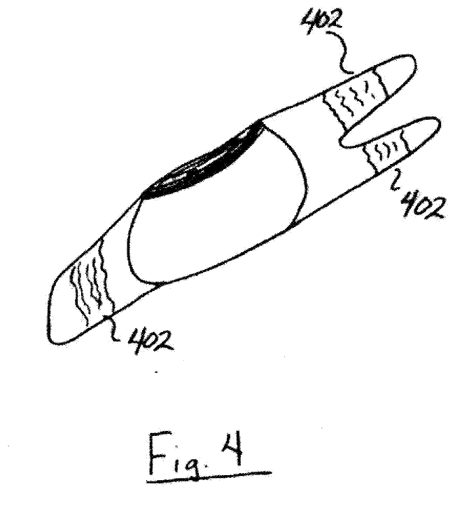

Some embodiments, as shown in FIG. 4, can also include an elastomeric region 402 of all or a portion of an extension 106. In alternative embodiments, a bag 102 can also include an elastomeric region 402 as all or part of a wall of a bag 102. An elastomeric region 402 can be made of a polymer, fabric, or any other known and/or convenient material.

FIG. 1b shows another embodiment of the present device wherein one extension 106 extends from one side of a bag 102, and at least two extensions 106 extend substantially from the opposite side of a bag 102. In some embodiments, as shown in FIG. 1b, extensions 106 can have texturing 110 on at least one surface of at least one extension 106. In some embodiments, at least two extensions 106 can extend separately from a bag 102, as shown in FIG. 1b, or can originate as a single extension and split off into a plurality of extensions, as shown in FIG. 4.

FIG. 1c shows another embodiment of the present device wherein one extension 106 extends from one side of a bag 102, and at least two extensions 106 extend substantially from the opposite side of a bag 102. In some embodiments, at least one extension 106 can attach to another extension 106 by sets of complementary protrusions 112 and apertures 114. In other embodiments, extensions 106 can attach to each other or the exterior surface of a bag 102 by hook-and-loop closures, hooks, snaps, buttons, or any other known and/or convenient connecting device.

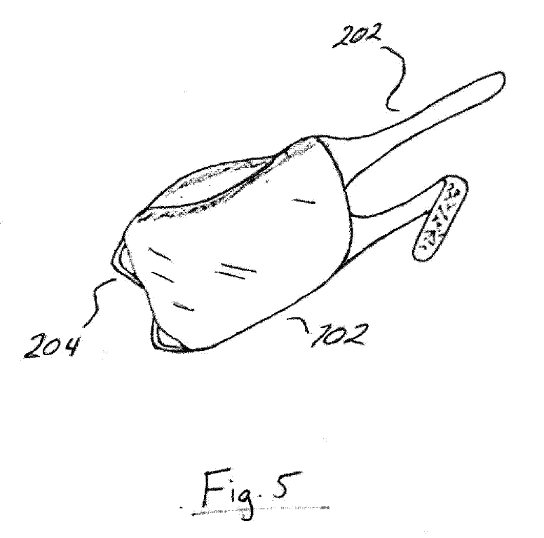

FIG. 5 shows another embodiment of the present device wherein at least one extension 106 can comprise at least one pair of an elongated strip 202 extending from one side of a bag 102 and a loop 204 extending from the opposite side. In some embodiments an elongated strip 202 can pass through a loop 204 such that the distal end of an elongated strip 202 can then be folded back to connect to the body of an elongated strip 202, thus securing an elongated strip 202 to a loop 204. An elongated strip 202 can connect to itself with adhesive, as shown in FIG. 5, or texturing, hook and loop, snaps, buttons or any other known and/or convenient device.

In use, a user can fill a bag 102 with ice or any other known and/or convenient cooling agent via an opening 104. A user can then close an opening 104 with a closure device 105. To secure the device around an injured body part, a user can then wrap at least one extension 106 around an injured body part so that an extension 106 can connect to another extension 106, the surface of a bag 102, or to a user's skin or clothing.

Although the invention has been described in conjunction with specific embodiments thereof, it is evident that many alternatives, modifications and variations will be apparent to those skilled in the art. Accordingly, the invention as described and hereinafter claimed is intended to embrace all such alternatives, modifications and variations that fall within the spirit and broad scope of the appended claims.

What is claimed is:

1. A disposable device for holding ice and securing it to a body part, comprising:
a bag having at least one resealable opening and an inner surface and an outer surface;
at least one extension attached to said bag, each having a top surface and a bottom surface.

2. The device of claim 1, wherein said bag is made of a waterproof material.

3. The device of claim 2, further comprising an adhesive material affixed to at least one surface of said at least one extension.

4. The device of claim 2, further comprising an adhesive material affixed to the outer surface of said bag.

5. The device of claim 2, further comprising texturing on at least one surface of at least one extension.

6. The device of claim 5, further comprising texturing on the outer surface of said bag.

7. The device of claim 2, wherein said at least one extension further comprises at least one set of closure devices.

8. The device of claim 2, wherein said at least one extension comprises at least one elongated member extending from one side of said bag paired with a loop extending from the opposite side of said bag.

9. The device of claim 8, wherein said at least one elongated member further comprises an adhesive on at least one surface of said at least one elongated member.

10. The device of claim 8, wherein said at least one elongated member further comprises texturing on at least one surface of said at least one elongated member.

11. The device of claim 8, wherein said at least one elongated member further comprises at least one set of closure devices on said at least one elongated member.
